(19) United States
(12) Plant Patent Application Publication
(10) Pub. No.: US 2002/0002721 P1
(43) Pub. Date: Jan. 3, 2002

(54) Gladiolus plant named 'Georgia Peach'

(76) Inventor: John O. Zipperer III, Quito (EC)

Correspondence Address: Laura G. Barrow, P.O. Box 215, Estero, FL, 33928-0215, US

(21) Appl. No.: 09/745,086

(22) Filed: Dec. 20, 2000

(51) Int. Cl. A01H 5/00

(52) U.S. Cl. PLT/301

(57) ABSTRACT

A new and distinct gladiolus cultivar, designated ‘Georgia Peach’, shown and described. Compared to the ‘Spic +Span’ variety, the ‘Georgia Peach’ cultivar produced three to four more flowers per stem, is four to six inches taller, and will maintain three to four more flowers in open bloom. The ‘Georgia Peach’ cultivar is able to maintain up to seven to eight flowers in open bloom simultaneously, beginning with a tight cut stem.

The present invention comprises a new and distinct cultivar of a Gladiolus plant referred to by the cultivar name ‘Georgia Peach.’

### BRIEF DESCRIPTION OF THE PHOTOGRAPHS

FIG. 1 is a photograph of a ‘Georgia Peach’ cultivar plant in bloom.

FIGS. 2-3 are photographs of the ‘Georgia Peach’ cultivar plant prior to blooming.

FIG. 4 is a photograph of a corm of the ‘Georgia Peach’ cultivar plant.

### ASEXUAL REPRODUCTION

The new cultivar was originated by the Applicant in a controlled proprietary breeding program in Ft. Myers, Fla. wherein selected gladiolus varieties were crossed. The female parent was a gladiolus variety named ‘Dr. Magee,’ characterized in part by having a small pink bloom, a short stem having a short flower head, and high resistance to Fusarium fungi species. The male parent was a red gladiolus variety named ‘T-111,’ characterized by having a long head, high bloom count, and good stem production. The seeds were planted in Ft. Myers, Fla., and the selection of ‘Georgia Peach’ cultivar was made in spring 1988. Asexual reproduction of the ‘Georgia Peach’ cultivar was achieved by collecting cormels from the first corm. All subsequent asexual reproductions of the ‘Georgia Peach’ are true to the original variety.

### BOTANICAL DESCRIPTION

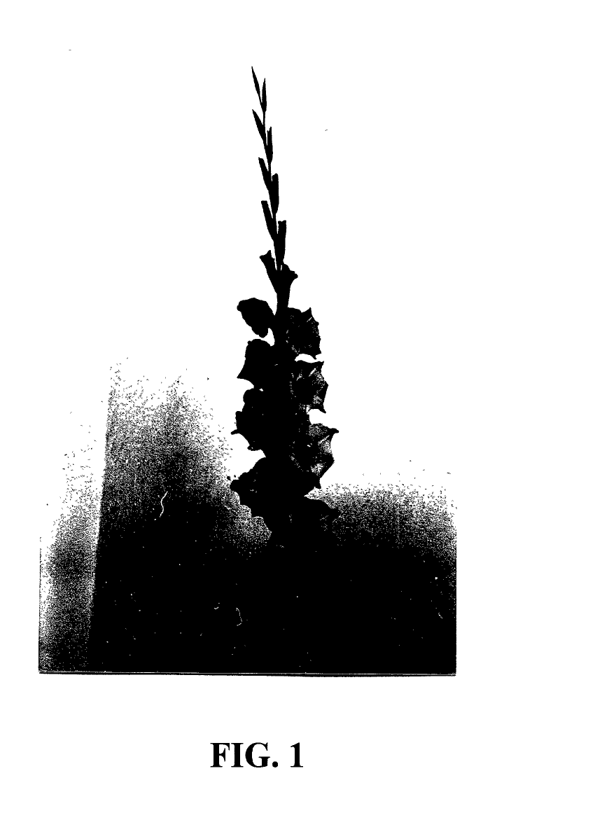

The accompanying color photograph (FIG. 1) shows the inflorescence and various stages of blooming of the ‘Georgia Peach’ cultivar plant.

The bud size of the ‘Georgia Peach’ cultivar is about 6 cm in length. The flowers of the ‘Georgia Peach’ cultivar each comprise four to five large petals with one to two small petals, all overlapping. The color of the corolla bloom is a strawberry red deep within the throat of the bloom, a cream color extending toward the outside of the bloom and terminating at the edge of the bloom with a peachy pink color. Small lines run up the middle of each petal. The diameter of the entire bloom is about 11 cm.

The spike of the plant is about 125 cm to 130 cm when grown from Jumbo size corms (1.75 inch and larger) in Ecuador. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are pink.

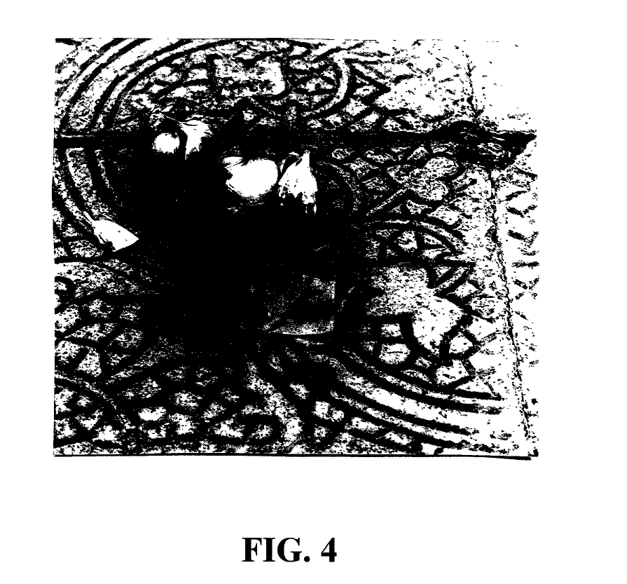

The corms of the ‘Georgia Peach’ cultivar are typical for the gladiolus and have a yellow color under the husk (FIG. 4).

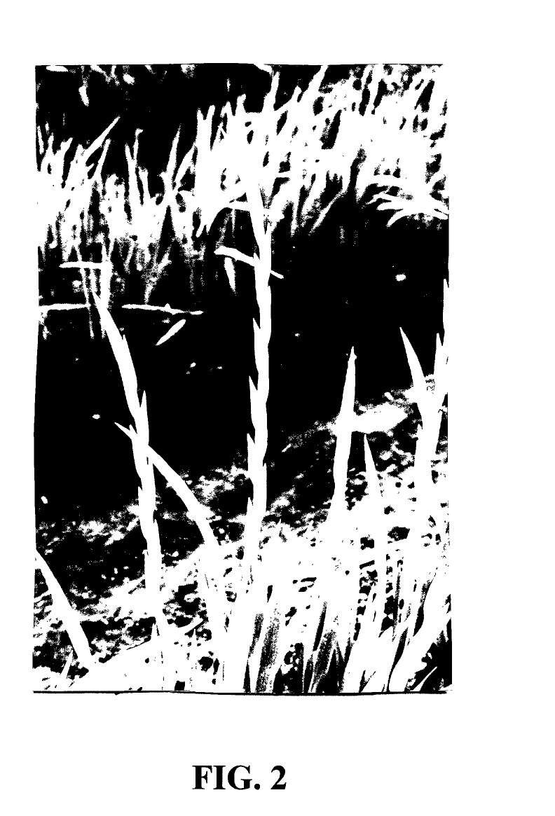

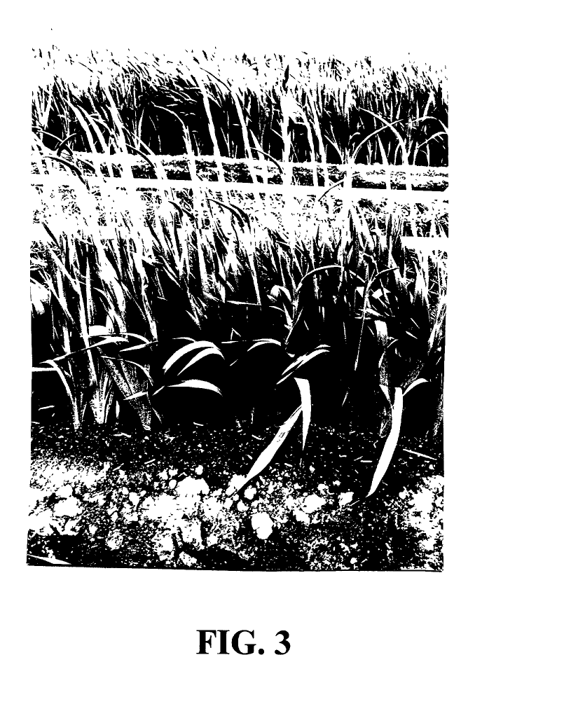

The leaf color of the ‘Georgia Peach’ cultivar is green and tapers to a point with parallel veins running the length of the leaf (FIGS. 2-3).

### GENERAL OBSERVATIONS

Compared to the gladiolus variety ‘Spic +Span,’ the ‘Georgia Peach’ cultivar produces three to four more flowers per stem, is four to six inches taller, and will maintain three to four more flowers in open bloom. The ‘Georgia Peach’ cultivar will, in fact, be able to maintain up to seven to eight flowers in open bloom simultaneously, beginning with a tight cut stem.

The foliage stands fairly straight during growth, and the cultivar harvests one week faster than the pink variety ‘Friendship.’

The ‘Georgia Peach’ cultivar is very resistant to attack by Fusarium and Curvalaria fungi species and does not attract worms or red spiders. The ‘Georgia Peach’ is also tolerant to high temperatures experienced in August through October in Florida as well as temperatures experienced in mid-winter.

The flowers open fast in the field and blooms well under short or along day lengths. The ‘Georgia Peach’ cultivar stems may be cut tight, shipped dry for a week, and still bloom well afterwards.

I claim:

1. A new and distinct gladiolus cultivar ‘Georgia Peach’, as shown and described herein.
